(19) United States
(12) Patent Application Publication
(10) Pub. No.: US 2024/0376436 A1
(43) Pub. Date: Nov. 14, 2024

(54) COMPOSITIONS AND METHODS FOR BIASING THE POLARITY OF ORGANOIDS

(71) Applicant: STEMCELL TECHNOLOGIES CANADA INC., Vancouver (CA)

(72) Inventors: Salvatore SIMMINI, Vancouver (CA); Juan HOU, Vancouver (CA); Georgios STROULIOS, Vancouver (CA); Tyler BROWN, Vancouver (CA)

(21) Appl. No.: 18/569,259

(22) PCT Filed: Jun. 14, 2022
(86) PCT No.: PCT/CA2022/050943

§ 371 Date: Dec. 12, 2023

Related U.S. Application Data
(60) Provisional application No. 63/286,173, filed on Dec. 6, 2021.
(60) Provisional application No. 63/210,248, filed on Jun. 14, 2021.

(51) Int. Cl. C12N 5/071

(52) U.S. Cl. CPC C12N 5/0688; C12N 2500/99; C12N 2501/15; C12N 2501/42; C12N 2513/00

(57) ABSTRACT

The present disclosure relates to media compositions and/or supplements to be added into an organoid medium, and to methods for generating apical-out organoids. Apical-out organoids exhibit an apical surface thereof facing the external environment, and an inwardly disposed basolateral surface. Apical-out organoids may be formed in organoid media comprise a basal medium and one or more of an inhibitor of notch signaling and an inhibitor of signaling through a TGF. Apical-out organoids formed in accordance with the disclosed methods or in the disclosed media may be used to assay host responses to pathogens.

### CROSS-REFERENCE TO RELATED APPLICATIONS

This disclosure claims the benefit of U.S. Provisional Appl. No. 63/210,248, filed Jun. 14, 2021 and U.S. Provisional Appl. No. 63/286, 173, filed Dec. 6, 2021, the contents of both of which are incorporated herein by reference in their entirety.

### FIELD

This disclosure relates to cell culture applications, and more specifically to cell culture applications using organoids, and still more specifically to cell culture applications wherein the polarity of organoids may be biased.

### BACKGROUND

Research, diagnosis and therapy with cells frequently relies on tissue and/or organ models, yet in many cases the existence of relevant, physiological models is lacking. In recent times, organoid technologies have shown promise as a relevant, physiological model for studying developmental biology, disease, susceptibility to viral infection, toxicology, sensitivity to drugs and compounds, personalized medicine, etc.

A challenge related to culturing organoids, particularly for epithelial organoids, and to using organoids in downstream assays is that they tend to form and grow with the apical surface facing a core (and in many cases a central lumen) of the organoid, while the basolateral surface faces the exterior environment. However, access to the apical side via the direct external environment is often helpful, and in some cases necessary, to conducting certain studies.

Accordingly, there is a need for improved media formulations and methods for generating so-called “apical-out” organoids.

### SUMMARY

This disclosure relates to media compositions and/or supplements to be added into a medium, and to methods for generating organoids, among which are included “apical-out” organoids. The media and methods disclosed herein produce such “apical-out” organoids with high efficiency.

In one broad aspect of this disclosure are provided organoid media for generating apical-out organoids, the media comprise a basal medium and one or more of an inhibitor of notch signaling, an inhibitor of transformation growth factor (“TGF”) signaling, and an agent that disrupts cytoskeletal structure. In one embodiment, the organoid medium is a pulmonary organoid medium.

In one embodiment, the organoid medium is serum-free.

In one embodiment, the organoid medium does not contain or come into contact with an added extracellular matrix or extracellular matrix protein. In one embodiment, the organoid does not form in the presence of an added extracellular matrix or extracellular matrix protein. In one embodiment, the population of cells, such as a population of pulmonary lineage cells, do not come into contact with an added extracellular matrix or extracellular matrix protein. In one embodiment, neither i) the organoid medium contains or comes into contact with an added extracellular matrix or extracellular matrix protein, nor does ii) the organoid form in the presence of an added extracellular matrix or extracellular matrix protein, nor do iii) the cells (e.g. pulmonary lineage cells) come into contact with an added extracellular matrix or extracellular matrix protein.

In embodiments where the organoid medium comprises an inhibitor of notch signaling, the inhibitor of notch signaling is a gamma secretase inhibitor.

In embodiments where the organoid medium comprises an inhibitor of signaling through a TGF, the inhibitor of signaling through a TGF is an inhibitor of TGFbeta signaling.

In embodiments where the organoid medium comprises a factor that modifies the cytoskeleton, the factor that modifies the cytoskeleton is an inhibitor of a Rho-associated protein kinase.

In one embodiment, the organoid medium does not include a factor that modifies the cytoskeleton. In one embodiment, the organoid medium does not include an inhibitor of a Rho-associated protein kinase.

In one broad aspect of this disclosure are provided methods of forming organoids in a culture, the methods comprising contacting a population of cells, such as a population of pulmonary lineage cells, with an organoid medium; and culturing the cells in the organoid medium. In one embodiment, the population of cells are cultured in the absence of an added extracellular matrix or extracellular matrix protein.

In one embodiment, the methods may further comprise, forming organoids wherein an apical surface of at least a portion of the organoids is in contact with the organoid medium.

In one embodiment, the population of pulmonary lineage cells are bronchial or nasal epithelial cells. In one embodiment, the population of pulmonary lineage cells are human or mouse.

In one embodiment, the methods further comprise aggregating the population of cells. In one embodiment, aggregating the population of cells is in the organoid medium. In one embodiment, aggregating the population of cells is for between 1 and 7 days. In one embodiment, aggregating comprises depositing between 10 and 2000 cells into a microwell device. In one embodiment, the deposited cells are single cells or comprised in clumps.

In one embodiment, the organoid medium is serum-free.

In one embodiment, the organoid medium comprises a basal medium.

In one embodiment, the organoid medium does not contain or come into contact with an added extracellular matrix or extracellular matrix protein. In one embodiment, the organoid does not form in the presence of an added extracellular matrix or extracellular matrix protein. In one embodiment, the cells, such as a population of pulmonary lineage cells, do not come into contact with an added extracellular matrix or extracellular matrix protein. In one embodiment, neither i) the organoid medium contains or comes into contact with an added extracellular matrix or extracellular matrix protein, nor does ii) the organoid form in the presence of an added extracellular matrix or extracellular matrix protein, nor do iii) the cells (e.g. pulmonary lineage cells) come into contact with an added extracellular matrix or extracellular matrix protein.

In one embodiment, the organoid medium comprises a basal medium supplemented with one or more of an inhibitor of notch signaling, an inhibitor of signaling through a TGF, and an agent that disrupts cytoskeletal structure.

In one embodiment, the organoid medium comprises an inhibitor of notch signaling. In one embodiment, the inhibitor of notch signaling is a gamma secretase inhibitor.

In one embodiment, the organoid medium comprises an inhibitor of signaling through a TGF. In one embodiment, the inhibitor of signaling through a TGF is an inhibitor of TGFbeta signaling.

In one embodiment, the organoid medium comprises a factor that modifies the cytoskeleton. In one embodiment, the organoid medium does not include a factor that modifies the cytoskeleton.

In one embodiment, the factor that modifies the cytoskeleton is an inhibitor of a Rho-associated protein kinase.

In one embodiment, culturing the population of pulmonary lineage cells in the organoid medium is under non-adherent conditions. In one embodiment, culturing is for between 5 and 25 days.

In one embodiment, at least 25% of cells of the portion of the organoids are ciliated. In one embodiment, at least 50% of cells of the portion of the organoids are ciliated.

In one embodiment, among at least 60% of the organoids the apical surface thereof is in contact with the organoid medium. In one embodiment, among at least 80% of the organoids the apical surface thereof is in contact with organoid medium.

In one embodiment, the organoids express one or more markers of ciliogenesis. In one embodiment, the one or more markers of ciliogenesis comprise FOXJ1 and/or TUBB4B.

In one embodiment, the organoids express one or more of TJP1 (i.e. ZO-1) and ACE2.

In one embodiment, the organoids do not express MUC5AC.

In one embodiment, the organoids support the replication of viruses.

In one embodiment, the methods may further comprise assessing organoid responses to one or more therapeutic agents, such as an anti-viral. In one embodiment, the methods may further comprise assessing organoid responses to one or more pathogens, such as virus or bacteria.

Thus advantages of methods and media disclosed herein include, but are not limited to, i) an ECM-free workflow may be superior to an ECM-removal workflow, in terms of the efficiency of generating apical-out organoids, ii) the efficient removal of dead, dying, or otherwise shed cells from the culture is facilitated because they do not accumulated in a lumen of an “apical-in” organoid, iii) a fast read-out of cilia beating with potential applications to study ciliopathies, iv) removing spatial limitations imposed by forming organoids within an extracellular matrix, and/or allowing for efficient scale-up, and v) studying viral infection and infectivity, and/or screening antiviral drugs.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows the results of initial characterizations of apical-out organoids generated in accordance with a method or using a medium of this disclosure. Organoid motility was measured by visually assessing ciliated organoids and scoring the motion of such ciliated organoids (panel A). The organoids from three different donors were assessed for motility across 6 consecutive passages beginning from passage 3. A histogram depicting the distributions of mean organoid diameters as measured at passage 3 from three different donors (panel B). 750 organoids were equally counted from 3 wells for each donor. A boxplot of the distributions of organoid diameters when organoids were formed from different starting cell numbers. Unshaded values correspond to a measurement taken at a relatively earlier time point, while shaded values correspond to a measurement taken after the organoids had been in suspension culture for at least a further week (panel C). The center line represents the median value, and the bounds of whiskers represent the maximum and minimum values. 75 organoids were measured for each condition (n=1).

FIG. 2 shows a comparison of apical-out airway organoid forming efficiency in different conditions. A bar graph showing for three different donors the efficiencies of forming apical-out organoids using either an ECM free workflow (ECM-free Ap-O AO) or a workflow wherein organoids are initially formed in a Matrigel™ dome and then removed from the Matrigel to attempt organoid inversion (ECM Ap-O AO) (panel A). Individual points represent technical replicates. Data are presented as mean±SD of 5 independent experiments. P value is calculated by unpaired Students' t-test (**=p<0.01, ***=p<0.001). A representative image of organoid fusing typically observed among organoids that could form in the ECM Ap-O AO workflow (scale bar=200 μm). White arrows indicate organoids that are fused but not completely merge (panel B).

FIG. 3 shows graphs quantifying the percentage of cells of organoids that are ciliated. The epithelial cells of a first donor (panels A) and B)) and of a second donor (panels C) and D)) were formed into apical-out organoids in accordance with this disclosure. In panels A) and C) the concentration of an inhibitor of Notch signaling was titrated while the concentration of an inhibitor of TGFbeta signaling was more-or-less constant. In panels B) and D) the concentration of an inhibitor of TGFbeta signaling was titrated while the concentration of an inhibitor of Notch signaling was more-or-less constant.

FIG. 4 shows graphs quantifying the number of apical-out organoids and the percentage of cells of the organoids that are ciliated. The epithelial cells of a first donor (panels A) and B)) and of a second donor (panels C) and D)) were formed into apical-out organoids in accordance with this disclosure. The effect of removing an inhibitor of Notch signaling on overall organoid number is shown in panels A) and C). The effect of removing an inhibitor of Notch signaling on the percentage of ciliated cells among the apical-out organoids is shown in panels B) and D).

FIG. 5 shows graphs quantifying the number of apical-out organoids and the percentage of cells of the organoids that are ciliated. The epithelial cells of a donor were formed into apical-out organoids in accordance with this disclosure. The effect of combining an inhibitor of Notch signaling with an inhibitor of TGFbeta signaling on overall organoid number (panel A) and the percentage of ciliated cells among the apical-out organoids (panel B) is shown.

FIG. 6 shows graphs quantifying the efficiency of apical-out organoid formation across multiple donors and multiple passages. Following formation of the apical-out organoids in accordance with this disclosure, the number of motile organoids was quantified across successive passages (panel A). During each passage, the dissociated cells were analyzed for the presence of cilia (panel B). Each point represents a different well of a 24-well plate (technical replicate). n=2 replicates per donor (up to p5).

FIG. 7 shows representative images of apical-out organoids formed in different conditions. An aggregate at p6 having been cultured in an organoid medium containing an inhibitor of TGFbeta signaling and Y-27632 shows patchy ciliation (panel A). An aggregate at p6 having been cultured in an organoid medium containing an inhibitor of Notch signaling shows ubiquitous ciliation (panel B). An aggregate at p6 having been cultured in an organoid medium containing an inhibitor of Notch signaling and an inhibitor of TGFbeta signaling shows ubiquitous ciliation (panel C).

FIG. 8 shows representative images of apical-out organoids stained for various markers. Apical-out organoids are positive for acetylated alpha tubulin (arrow) and ZO-1 (arrowhead), but are negative for MUC5AC (panel A). Apical-out organoids are positive for acetylated alpha tubulin (arrow) and ACE2 (arrowhead) (panel B).

FIG. 9 shows the results of qRT-PCR experiments for various markers of pulmonary lineage cells. Gene expression among the cells of apical-out organoids formed in accordance with this disclosure (AOAO) was compared to various controls: donor cells cultured under air-liquid interface conditions (ALI) in PneumaCult ALI (STEMCELL Technologies); donor cells formed into organoids using the PneumaCult Airway Organoid Kit (AOK) (STEMCELL Technologies); and donor cells expanded in PneumaCult ExPlus (Ex+) (STEMCELL Technologies).

FIG. 10 shows graphs quantifying the number of apical-out organoids and the percentage of cells of the organoids that are ciliated across multiple passages. Apical-out organoids were formed from four donors (panels A) and B); panels C) and D); panels E) and F); and panels G) and H)) in accordance with this disclosure, in either an organoid medium containing inhibitors of Notch signaling and TGFbeta signaling (AOAO) or in a control organoid medium containing an inhibitor of TGFbeta signaling and Y-27632 (Control). The number of apical-out organoids formed from four donor samples in the two media are shown in panels A), C), E), and G). The percentage of ciliated cells among the apical-out organoids formed from four donor samples in the two media are shown in panels B), D), F), and H).

### DETAILED DESCRIPTION

This disclosure relates to media compositions and/or supplements to be added into a medium, and to methods for generating “apical-out” organoids. The media and methods disclosed herein produce such “apical-out” organoids with high efficiency.

Where used in this disclosure, the term “organoid” refers to a multicellular structure that may be generated ex vivo and that exhibits higher-level organization, reminiscent of the organization observed in a corresponding tissue. Organoids corresponding to various tissue types may be formed using, for example, kits and protocols commercialized by STEMCELL Technologies. This disclosure primarily focuses on epithelial organoids, but is not necessarily limited to only epithelial organoids. Examples of epithelial tissues that may be formed into the organoids of this disclosure (in accordance with the media and methods disclosed herein) include pulmonary organoids, whether corresponding to the proximal airway, the distal airway, or both.

Where used in this disclosure, the term “pulmonary lineage cells” refers to one of various types of cells that may be isolated from the nasal cavity, the trachea, or lung tissue, including the bronchi, bronchioles, or alveoli. The term “pulmonary lineage cells” may also refer to one of various types of cells differentiated from pluripotent stem cells (PSC), including embryonic stem cells or induced pluripotent stem cells. If differentiated from PSC, the pulmonary lineage cells may share one or more common features with a corresponding or closely corresponding primary (i.e. patient derived) cell type. The pulmonary lineage cells of this disclosure may be from any species, but are preferably mammalian, and more preferably are human or mouse. In one embodiment, the pulmonary lineage cells are human pulmonary epithelial cells. In one embodiment, the pulmonary lineage cells are human bronchial epithelial cells. In one embodiment, the pulmonary lineage cells are basal cells.

Where used in this disclosure, the term “organoid medium” or “organoid media” refers to a cell culture medium comprising a basal medium that is appropriately supplemented to form the organoids of this disclosure. Indeed, basal media are well known in the art and are routinely formulated to include one or more of salt(s), amino acid(s), carbohydrate(s), buffer(s), trace elements, etc. Examples of commercially available basal media include DMEM, Adv-DMEM, DMEM/F-12, RPMI, Iscoves, and various others marketed specifically for the culture of epithelial cells. The specific supplementation of the basal medium—with for example cytokines, growth factors, small molecules, serum/albumin/serum replacement, lipids, etc.—will depend on the application to which the organoid medium is put. In embodiments where the organoids are pulmonary organoids, it is common to include one or more of: a mitogen; a fibroblast growth factor; an activator and/or an inhibitor of signaling through a bone morphogenic protein; an activator and/or an inhibitor of signaling through a wnt; a vitamin A analogue, precursor or metabolite/derivative; and an activator and/or an inhibitor of signaling through transformation growth factor (e.g. transformation growth factor beta). In one embodiment, an organoid medium comprises a basal medium and an inhibitor of notch signaling and/or an inhibitor of signaling through a TGF.

Where used in this disclosure, the term “apical-out” refers to an organoid, and in some embodiments an epithelial organoid, wherein the apical surface is in direct contact with the external environment (i.e. a cell culture medium). Among apical-out organoids, some or all of such organoids may comprise a lumen, in which case a basolateral side thereof is in direct contact with the lumen. However, some apical-out organoids may not comprise a lumen, but may rather exhibit a compact/dense conformation, in which case the basolateral side thereof may be comprised in or adjacent a central core of such apical-out organoids.

### Organoid Media

In one aspect of this disclosure are provided organoid media for generating apical-out organoids. In a preferred embodiment, the organoid media are for generating apical-out pulmonary organoids, such as starting from pulmonary epithelial cells. Thus, where an organoid medium is used to generate apical-out pulmonary organoids, it may be characterized as a pulmonary organoid medium.

The organoid medium may comprise an inhibitor of notch signaling. In one embodiment, the inhibitor of notch signaling is a gamma secretase inhibitor. Examples of inhibitors of notch signaling include DBZ, DAPT, Compound E, Compound W, SAHM1, and FLI 06. In one embodiment, the inhibitor of notch signaling is DAPT.

If present in the organoid medium, the inhibitor of notch signaling will be present at an effective concentration while also being present at a concentration that does not result in any or in significant levels of cell toxicity. In one embodiment, a concentration of the inhibitor of notch signaling ranges between about 1 nM and 1 mM, between about 5 nM and 500 μM, between about 10 nM and 200 μM, between about 50 nM and 100 μM, between about 100 nM and 50 μM, or between about 0.5 μM and 20 μM.

In one embodiment, the organoid medium comprises an inhibitor of signaling through a TGF. In one embodiment, the TGF is TGFbeta. In one embodiment, the inhibitor of signaling through a TGF is a natural inhibitor, such as an endogenous protein. In one embodiment, the inhibitor of signaling through a TGF is synthetic inhibitor, such as a small molecule. Examples of inhibitors of signaling through a TGF include A83-01, A77-01, SB 431542, LY364947, and LDN 214117. In one embodiment, the inhibitor of signaling through a TGF is A83-01. In one embodiment, the inhibitor of signaling through a TGF is A77-01. In one embodiment, the inhibitor of signaling through a TGF is SB 431542.

If included in the organoid medium, the inhibitor of signaling through a TGF will be present at an effective concentration while also being present at a concentration that does not result in significant levels of cell toxicity. In one embodiment, a concentration of the inhibitor of signaling through a TGF ranges between about 1 nM and 1 mM, between about 5 nM and 500 μM, between about 10 nM and 200 μM, between about 50 nM and 100 μM, between about 100 nM and 50 μM, or between about 0.5 μM and 20 μM.

In one embodiment, the organoid medium includes one or both of an inhibitor of notch signaling (e.g. a gamma secretase inhibitor) and an inhibitor of signaling through a TGF. In one embodiment, the organoid medium includes only one of an inhibitor of notch signaling (e.g. a gamma secretase inhibitor) or an inhibitor of signaling through a TGF.

In one embodiment, the organoid medium does not include an extracellular matrix or an extracellular matrix protein. Thus, in one embodiment, the organoid medium does not include (or contain or come into contact with) an added extracellular matrix or extracellular matrix protein. Accordingly, the organoid medium does not require Matrigel™ or any other extracellular matrix to form the apical-out organoids.

In one embodiment, the organoid medium does not contain or come into contact with an extracellular matrix or extracellular matrix protein (unless naturally produced by the cells being cultured). In one embodiment, the organoid does not form in the presence of an exogenously added extracellular matrix or extracellular matrix protein. In one embodiment, the cells (e.g. the pulmonary lineage cells) do not come into contact with an exogenously added extracellular matrix or extracellular matrix protein(s). In one embodiment, neither the pulmonary lineage cells, the aggregated pulmonary lineage cells, nor the organoids come into contact with an exogenously added extracellular matrix protein.

In one embodiment, the organoid medium is serum-free. In some embodiments of a serum-free medium, the medium may comprise an albumin or a different serum replacement. If the organoid medium includes an albumin, it may be isolated from serum, and more specifically from animal serum. Or, the albumin may be recombinant and expressed in a cell line, such as a bacterial, fungal, plant, or animal cell line.

In one embodiment, the organoid medium comprises a factor that modifies the cytoskeleton. In one embodiment, the organoid medium does not comprise a factor that modifies the cytoskeleton. Examples of factors that modifies the cytoskeleton (included or specifically excluded from the organoid medium) include a Rho-associated protein kinase inhibitor, a p21-activated kinase (PAK) inhibitor, or a myosin II inhibitor. Examples of a Rho-associated protein kinase inhibitor include Y-27632, SR 3677, thiazovivin, HA1100 hydrochloride, HA1077 and GSK-429286. An example of a PAK inhibitor is IPA3. An example of a myosin Il inhibitor is blebbistatin. In a preferred embodiment, the organoid medium does not include a factor that modifies the cytoskeleton.

In one embodiment, the organoid medium includes one of, one or more of, two of more of, or each of an inhibitor of notch signaling (e.g. a gamma secretase inhibitor), an inhibitor of signaling through a TGF, and a factor that modifies/disrupts cytoskeletal structure.

### Methods

In one aspect of this disclosure are provided methods for generating organoids, a proportion of which are apical-out organoids. In a preferred embodiment, the methods are for generating apical-out pulmonary organoids, such as starting from pulmonary epithelial cells.

Methods of forming organoids (e.g. apical-out organoids) in a culture will comprise contacting a population of cells (e.g. pulmonary lineage cells) with an organoid medium. In one embodiment, an organoid medium of this disclosure comprises a basal medium and one or both of an inhibitor of notch signaling and an inhibitor of signaling through a TGF. In one embodiment, an organoid medium of this disclosure comprises a basal medium and both of an inhibitor of notch signaling and an inhibitor of signaling through a TGF. In one embodiment, an organoid medium of this disclosure comprises a basal medium and only one of an inhibitor of notch signaling or an inhibitor of signaling through a TGF. In one embodiment, an organoid medium of this disclosure comprises a basal medium and one or more of an inhibitor of notch signaling, an inhibitor of signaling through a TGF includes both, and an agent that disrupts cytoskeletal structure. In any case, an organoid medium, such as a pulmonary organoid medium, used in the methods of this disclosure may be formulated as described hereinabove.

Methods of forming organoids (e.g. apical-out organoids) in a culture will comprise culturing the population of cells (e.g. pulmonary lineage cells) in the organoid medium. In one embodiment, culturing the population of cells (e.g. pulmonary lineage cells) in the organoid medium occurs in the absence of an added extracellular matrix or extracellular matrix protein.

The methods will yield apical-out organoids wherein an apical surface of at least a portion of the apical-out organoids faces away from a core (e.g. a lumen) thereof, but in any event the apical surface of apical-out organoids will be in direct contact with the external environment. In one embodiment, a majority of the formed organoids (i.e the portion) exhibit an apical-out morphology/organization. In one embodiment, among at least 60% of the organoids the apical surface faces away from the core (or, lumen). In one embodiment, among at least 80% of the organoids the apical surface faces away from the core (or, lumen). In one embodiment, about 90% or more of the organoids exhibit an apical surface that faces away from the core (or, lumen) thereof.

In one embodiment, at least 25% of cells of the apical-out organoids are ciliated. In one embodiment, at least 50% of cells of the apical-out organoids are ciliated.

In one embodiment, the methods further comprise aggregating the population of cells (e.g. pulmonary lineage cells). In one embodiment, the methods further comprise aggregating the population of cells (e.g. pulmonary lineage cells) prior to or at the same time as the contacting step.

The cells may be aggregated using any known means. Although it is variously reported that cells may form into aggregates when deposited into and become settled within a well of certain types of 96-well plate, this approach results in high variability in terms of aggregate size and the number of aggregates formed per well. A better approach may be to use the AggreWell™ microwell device, in order to ensure the formation of a single aggregate per well and to yield more-or-less uniformly sized aggregates.

In one embodiment, aggregating the cells (e.g. pulmonary lineage cells) occurs in the absence of an added extracellular matrix or extracellular matrix protein(s). In one embodiment, the aggregating and aggregated cells (e.g. pulmonary lineage cells) do not contact or come into contact with an added extracellular matrix or extracellular matrix protein(s).

In one embodiment, aggregating the population of cells (e.g. pulmonary lineage cells, such as isolated basal cells) comprises bringing between 10 and 2000 cells into close proximity in a common well. In one embodiment, the number of cells formed into an aggregate is between 50 and 150 cells. In a specific embodiment, aggregating the population of pulmonary lineage cells comprises depositing between 10 and 2000 pulmonary lineage cells per well of a microwell device. In one embodiment, the deposited cells are single cells or comprised in clumps, or a mixture thereof. Interestingly, aggregates/organoids initially formed from different cell numbers may undergo cell “shedding” when cultured in suspension, which could result in a convergence to more-or-less equivalent diameters, regardless of an initial organoid diameter, after 5 or more days in suspension culture.

In one embodiment, the population of pulmonary lineage cells are aggregated in an organoid medium of this disclosure. In one embodiment, the population of pulmonary lineage cells are aggregated in an organoid medium for between 1 and 7 days. In one embodiment, the population of pulmonary lineage cells are aggregated in an organoid medium for 4 days+2 days.

In one embodiment, culturing the population of cells (e.g pulmonary lineage cells) is under non-adherent conditions. In one embodiment, the aggregating step is performed under non-adherent conditions, such as through using ultra low attachment 96-well plates or by coating the laboratory ware used with the anti-adherence solution provided with AggreWell™ plates. In one embodiment, the contacting step is performed under non-adherent conditions. In one embodiment, the contacting and the culturing steps are under non-adherent conditions, as well as the aggregating step, if taken.

In one embodiment, culturing the population of cells (e.g pulmonary lineage cells) in the organoid medium and in the absence of an added extracellular matrix or extracellular matrix protein(s), is for a time sufficient to yield apical-out organoids. In one embodiment, culturing is for between 5 and 25 days. In one embodiment, culturing is for 9 days+3 days. In one embodiment, culturing is for 15 days. In embodiments where the population of pulmonary lineage cells are aggregated prior to or during the contacting step, the culturing step may be shortened if it is apparent that apical-out organoids are emerging.

As discussed with regard to organoid media of this disclosure, such organoid media do not include an extracellular matrix or an extracellular matrix protein. In one embodiment, the organoid medium does not contain or come into contact with an extracellular matrix or extracellular matrix protein (unless naturally produced by the cells being cultured). Thus, in one embodiment, the organoid medium does not include (or contain or come into contact with) an added extracellular matrix or extracellular matrix protein. Accordingly, the organoid medium does not require Matrigel™ or any other extracellular matrix to form the apical-out organoids.

Similar, the contacting, culturing, and if applicable aggregating, steps are performed in the absence of an added extracellular matrix or extracellular matrix protein. Thus, the laboratory ware used during these steps is not coated with an extracellular matrix or an extracellular matrix protein. Accordingly, extracellular matrices commonly used for organoid formation, such as Matrigel™ or others, are not needed to practice the methods of this disclosure.

A population of pulmonary lineage cells may be expanded and passaged using a commercially available kit, such as PneumaCult™ Ex or PneumaCult™ Ex Plus (STEMCELL Technologies). At each passage some or all of the expanded cells may be used in the methods disclosed herein to obtain apical-out organoids. If not all of the expanded cells are used to generate apical-out organoids, then the remaining cells may be passaged to enable the formation of successive “generations” of apical-out organoids. The population of pulmonary lineage cells expanded and passaged, as by PneumaCult™ Ex or PneumaCult™ Ex Plus, may be propagated for 5 or more passages, or 8 or more passages.

Apical-out organoids that have been formed in accordance with this disclosure may express one or more markers of ciliogenesis. For example, the one or more markers of ciliogenesis may comprise FOXJ1 and TUBB4B.

Apical-out organoids that have been formed in accordance with this disclosure may express one or more markers relevant to their function or identity. For example, the one or more marker relevant to their function or identity may comprise TJP1 (i.e. ZO-1) and ACE2.

Apical-out organoids that have been formed in accordance with this disclosure may be void of MUC5AC expression, or express MUC5AC below detectable levels.

Apical-out organoids that have been formed in accordance with this disclosure may support the replication of viruses, such as respiratory or other viruses. In one embodiment, the methods may further comprise assessing organoid responses to one or more therapeutic agents. In one embodiment, the methods may further comprise assessing organoid responses to one or more pathogens, such as virus or bacteria.

The following non-limiting examples are illustrative of the present disclosure.

### EXAMPLES

### Example 1

### Culturing and Expanding Starting Cells

Primary normal human bronchial epithelial cells (hBECs) were commercially sourced, such as from LONZA or EPITHELIX SARL. In the experiments described herein, all cells were collected from non-smoking, healthy donors. Optionally, hBECs were seeded at passage one into T25 cell culture tissue flasks in PneumaCult™ EX-PLUS (STEMCELL Technologies, catalogue #05040) and incubated at 37° C. and 5% CO₂ in accordance with the manufacturer's protocol. Once cells reached 50-60% confluency, they were dissociated using the Animal Component-Free (ACF) Cell Dissociation kit (STEMCELL Technologies, catalogue #05426).

### Example 2

### Laboratory Ware Preparation

Plates used in downstream assays were coated with Anti-Adherence Rinsing Solution (STEMCELL Technologies, catalogue #07010). Briefly, 500 μL of Anti-Adherence Rinsing Solution was added to each well to be used and the plate was centrifuged for 10 min at 1300 g. Anti-Adherence Rinsing Solution was then removed, and the well was washed once with 1 mL DMEM. The wells were used directly, or they were stored for up to a week at 37° C. after adding 500 μL DMEM.

### Example 3

### Forming Aggregates

Cells were cultured and dissociated in accordance with Example 1, and seeded into Aggrewell™ 400 plates (STEMCELL Technologies) that were prepared in accordance with Example 2. Aggregates were generated by seeding roughly 1.2×10⁵ cells per well (100 cells per microwell) of an Aggrewell™ 400 plate. The cells were seeded in 500 μL of apical-out organoid medium comprising a basal medium and an inhibitor of notch signaling. The AggreWell™ plate was centrifuged for 3 min at 100 g in order to sediment the cells to the bottom of the microwell. The plates were then incubated for 24 h to 144 h at 37° C. and 5% CO₂.

### Example 4

### Suspension Culture of Apical-Out Organoids

After the aggregates were formed and sufficiently matured in accordance with Example 3, 500 μL of fresh apical-out airway organoid medium was added to each well. The aggregates were then resuspended using a P1000 pipette and each well distributed to two wells of a 24-well plate (prepared as described in Example 2). The aggregates were incubated under non-adherent conditions in apical-out airway organoid medium for up to 15 days. A partial medium change was performed every second day.

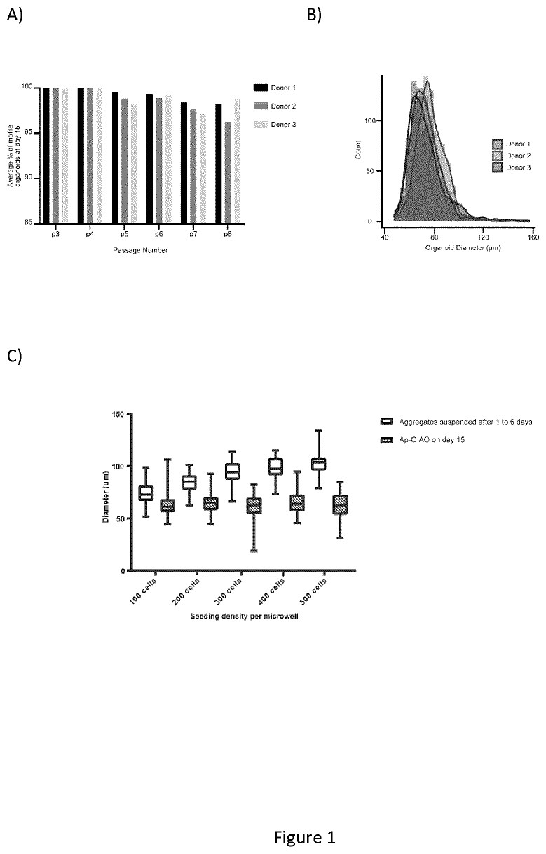

Day 15 organoids were assessed for their motility in suspension, as a proxy for beating cilia facing the culture environment. Essentially all of the apical-out organoids formed from three different donors exhibited motility when assessed at numerous sequential passages (FIG. 1A). Thus, the tested conditions for forming apical out organoids appeared to be highly efficiency. Day 15 organoids were also assessed for size (i.e. mean organoid diameter), and the apical-out organoids formed from three donor samples exhibited a tight size distribution (FIG. 1B). Specifically, average diameters of 72.6+6.7 μm (Donor 1), 77.3+4.7 μm (Donor 2), and 73.6+0.3 μm (Donor 3) were observed for the different populations of organoids. Interestingly, even when seeding more than 100 cells per microwell (e.g. 200, 300, 400, or 500 cells/well), the mean diameters of such organoids measured at day 15 were essentially identical to that of organoids generated from 100 cells per microwell (FIG. 1C).

### Example 5: Dissociating apical-out organoids

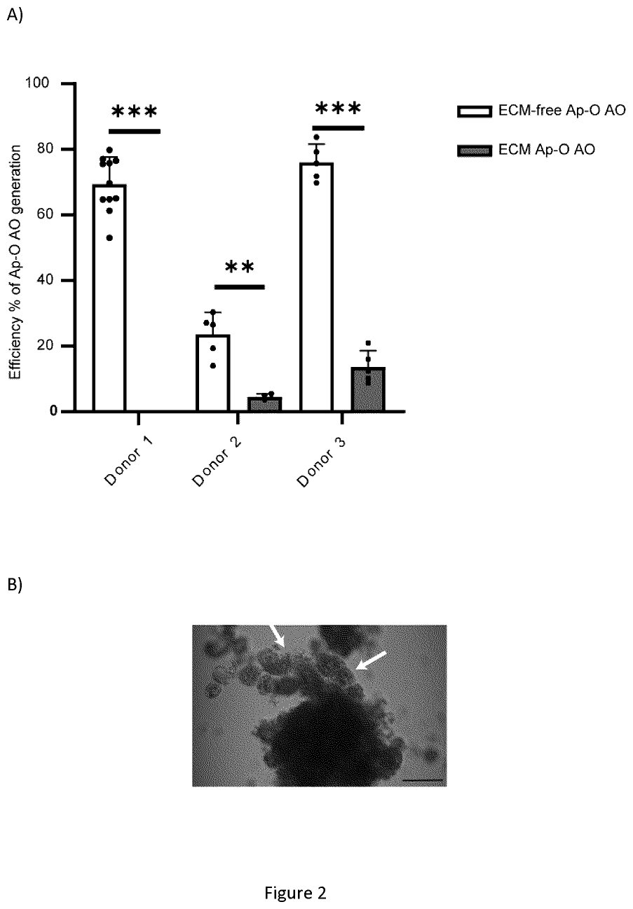

The efficiency of apical-out airway organoid formation was compared between organoids formed in accordance with Example 4 (i.e. ECM-free conditions) and organoids generated by removal from ECM conditions (i.e. ECM-removal conditions). Briefly, ECM-removal conditions involved generating organoids using PneumaCult™ Airway Organoid Kit (STEMCELL Technologies) by seeding 2500 human bronchial epithelial cells in a Matrigel™ dome and culturing for 7 days. The Matrigel was removed after 7 days by incubation in the Gentle Cell Dissociation Reagent (STEMCELL Technologies) at 4° C. on a shaker for 1 hour. The data show that apical-out airway organoids either failed to form or formed with poor efficiency in the ECM-removal condition, and that the efficiencies were significantly improved in the ECM-free workflow disclosed herein (FIG. 2A). Further, under the ECM-removal conditions, multiple of the apical-out airway organoids that could form tended to fuse into larger organoids (FIG. 2B). Overall, the ECM-free workflow shows marked improvements over the ECM-removal workflow.

### Example 6

### Dissociating Apical-Out Organoids

Apical-out airway organoids formed in accordance with Example 4 were harvested on day 15, transferred to a 15 ml tube, and centrifuged at 150 g for 5 min. The supernatant was removed, and the organoids were washed once with DMEM. The washed organoids were resuspended in ACCUTASE (STEMCELL Technologies) and were incubated for 15 minutes at room temperature, before being dissociated to single cells by pipetting vigorously with a P1000 pipette. The single-cell suspension was diluted 1:1 with trypan blue and was then loaded to a Hemocytometer where the cells were counted manually. At the time the dissociated cells are counted, the number of ciliated cells can also be determined by visual inspection.

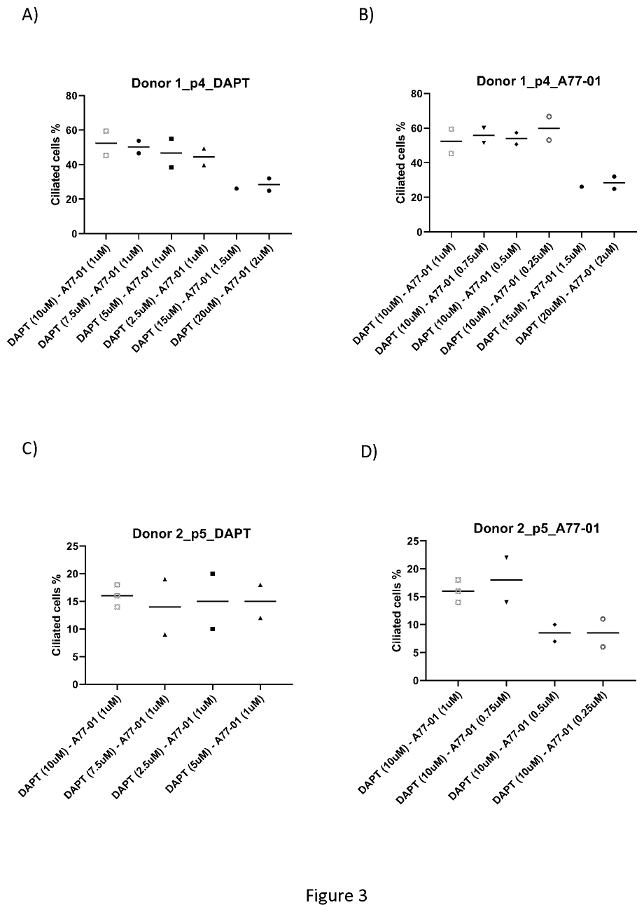

In one experiment, apical-out organoids were formed from two donor samples in apical-out media formulated with different concentrations of an inhibitor of notch signaling and an inhibitor of TGFbeta signaling. The apical-out organoids were dissociated, and the cells were inspected for the number of ciliated cells (FIG. 3). For the first donor (panels A and C), all conditions yielded 20% or more ciliated cells, but it appeared for this donor that the highest concentrations of both DAPT and A77-01 resulted in a decreased percentage of ciliated cells. Building on the results from the first donor, only the best performing conditions were tested on cells from the second donor (panels B and D). It appeared that lower concentrations of A77-01 were detrimental to the emergence of ciliated cells. Thus, it appeared that a concentration of 2.5 μM to 10 μM for the inhibitor of notch signaling, and a concentration between 0.5 μM and 1.5 μM for the inhibitor of signaling through a TGF worked reasonably well for efficient generation of ciliated cell percentage.

### Example 7

### Characterizing Apical-Out Organoids

Fully mature organoids, formed in accordance with Example 4, were imaged using a standard microscope camera. For example, the organoids may be imaged using a Leica DMi8 or an EVOS M500. The number of organoids was quantified and the percentage of motile organoids (i.e. % ciliated organoids) was assessed by visual inspection and by manually counting organoids that displayed beating cilia on the outer side.

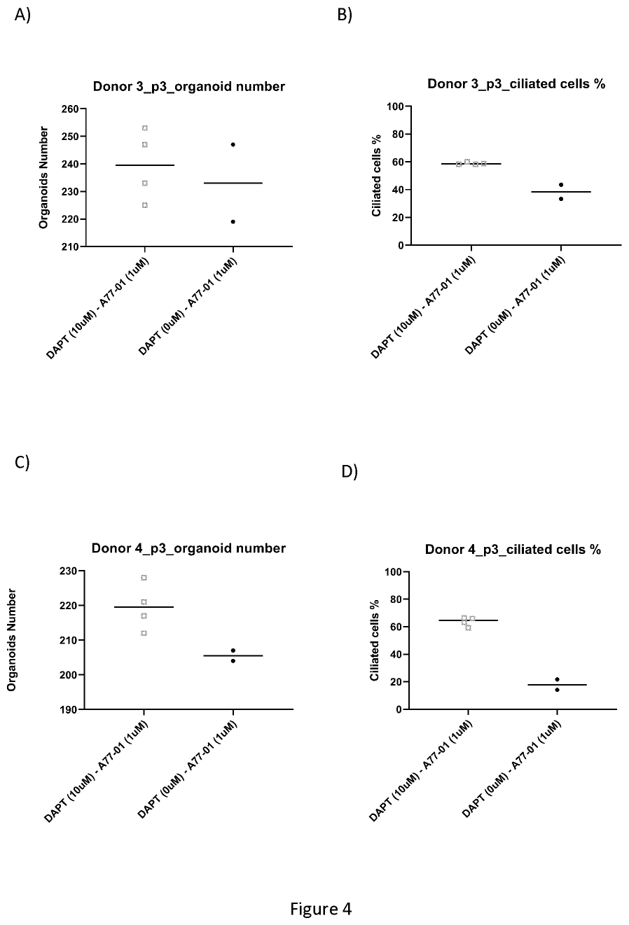

Cells from two different donors were formed into apical-out organoids using apical-out organoid media formulated with either an inhibitor of signaling through a TGF alone or in combination with an inhibitor of notch signaling (FIG. 4). While the presence or absence of the inhibitor of notch signaling (in combination with the inhibitor of signaling through a TGF) did not appear to markedly impact the number of organoids obtained (FIGS. 4A and 4C), absence of the inhibitor of notch signaling had a significant impact on the percentage of ciliate cells among the organoids (FIGS. 4B and 4D). Accordingly, an inhibitor of notch signaling appears important to the efficient differentiation of apical-out organoids.

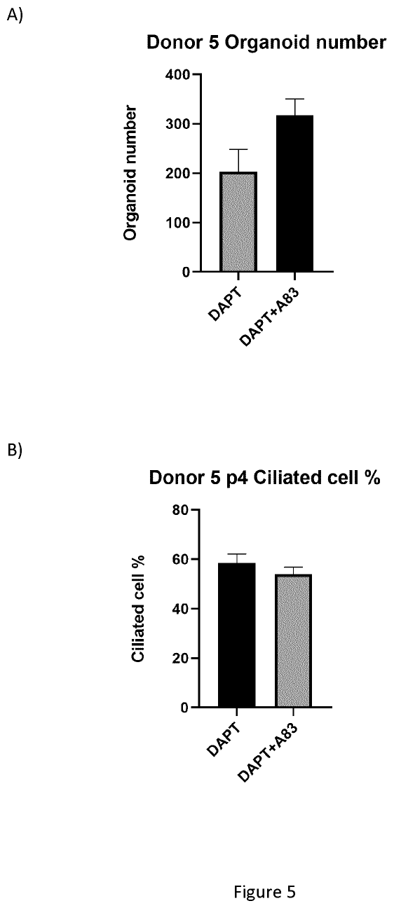

Conversely, the role of the inhibitor of signaling through a TGF was queried by either including or omitting it from an apical-out organoid medium (including an inhibitor of notch signaling) (FIG. 5). It appeared that including the inhibitor of signaling through a TGF may increase the number of output organoids (FIG. 5A), its absence appeared to have no impact or a positive impact on the percentage of ciliated cells among the organoids (FIG. 5B).

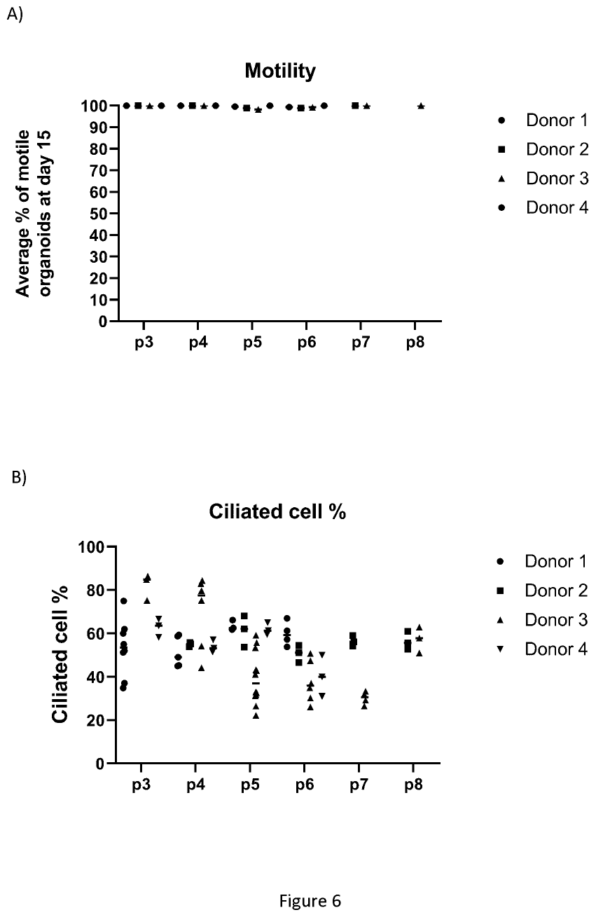

Since inclusion of an inhibitor of signaling through a TGF appeared important for increasing the number of output organoids, and inclusion of an inhibitor of notch signaling appeared important for differentiation of the donor pulmonary lineage cells, both were included in apical-out organoid media used for subsequent studies. Following the formation of apical-out organoids from the pulmonary lineage cells from four different donors in accordance with Example 4, visual inspection under a microscope revealed that virtually all of the formed organoids were motile, indicating sufficient ciliation of the organoids (FIG. 6A). The pulmonary lineage cells were passaged up to 8 times, depending on the donor and cell number, and at each passage apical-out organoids could be formed with high efficiency (FIG. 6A). The organoids from each “passage” were dissociated and the cells were assessed for percentage ciliation (FIG. 6B). Appreciable proportions of the cells exhibited ciliation.

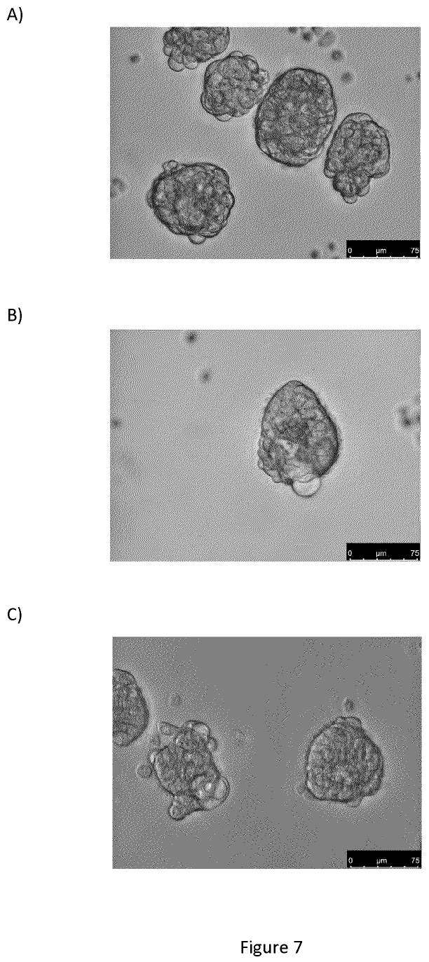

Representative images of apical-out organoids generated from passage 6 pulmonary lineage cells from a donor are shown in FIG. 7. Whereas patchy ciliation was observed among organoids formed in a medium comprising an inhibitor of TGFbeta signaling and Y-27632 (FIG. 7A), ubiquitous ciliation was observed for apical-out organoids formed in an apical-out medium including an inhibitor of notch signaling alone (FIG. 7B) or in combination with an inhibitor of signaling through a TGF (FIG. 7C).

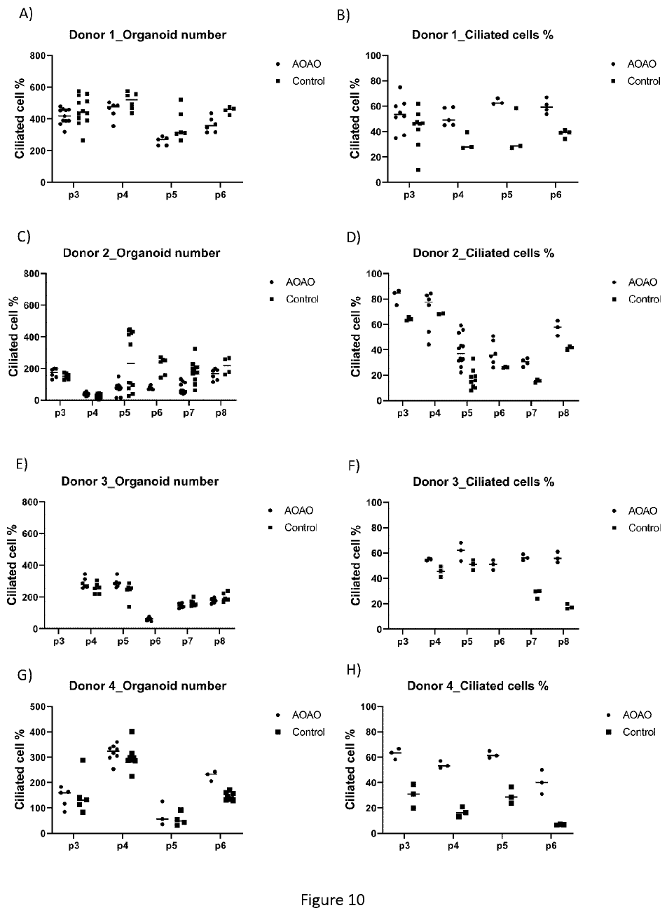

For comparison, organoids formed from pulmonary lineage cells of four different donors in the medium comprising an inhibitor of TGFbeta signaling and Y-27632 (“Control”) were compared head-to-head to apical-out organoids of this disclosure with regard to the number of output organoids (FIGS. 10A, 10C, 10E, and 10G) and percentage of ciliated cells (FIGS. 10B, 10D, 10F, and 10H). While the output organoid number was more or less equivalent between the Control and the apical-out conditions, it was quite clear that the apical-out condition was superior to the Control condition for differentiating the organoids to have increased percentages of ciliated cells.

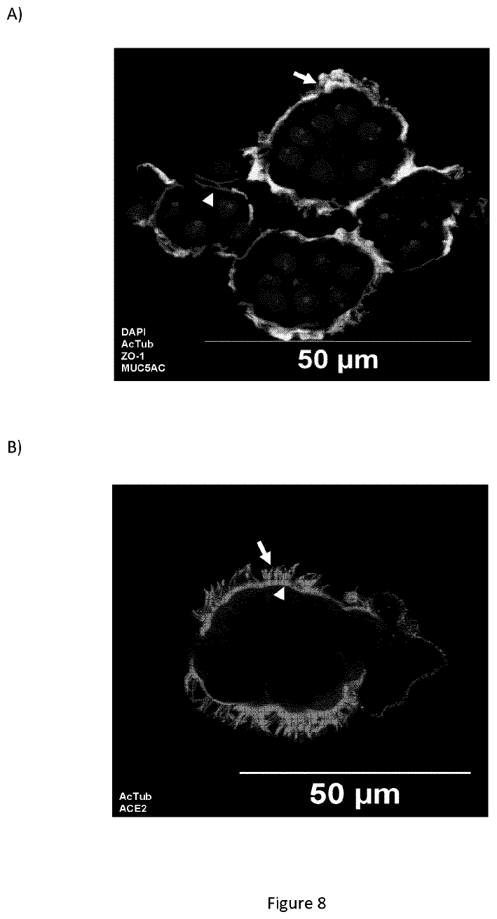

Organoids were stained in accordance with following generalized protocol. Apical-out organoids were fixed in 4% Paraformaldehyde, and they were subjected to antigen retrieval before being permeabilized with 1% Triton X-100 in PBS. Permeabilized organoids were blocked with 0.1% BSA/5% Normal Goat serum in PBS +0.1% Tween-20+0.2% Triton X-100 (PBSTT). Primary antibodies were diluted in PBSTT and incubated with the cells at room temperature in a tube with gentle agitation for 4 days. Certain airway epithelial markers were tested including Acetylated α-tubulin (Sigma, cat #T7451, 1:500), TMPRSS2 (Sigma, cat #MABF2158), ACE2 (Abcam, cat #ab15348), MUC5AC (Abcam, cat #ab212636). Respective secondary antibodies were used for the primary antibodies. Cells were washed with PBSTT and the respective secondary antibody was incubated at room temperature in a tube with gentle agitation for 3 days. Cells were washed again with PBSTT before staining with 4′, 6-diamidino-2-phenylindole (DAPI) and imaged with LEICA SP8. Representative images are shown in FIG. 8.

Total RNA was isolated from apical-out airway organoids using the Qiagen Easy RNA mini kit (Qiagen) as per the manufacturer's protocol. 500 ng of RNA was DNase treated (Invitrogen) as per the manufacturer's protocol and then reverse-transcribed to cDNA using SuperScript III (Invitrogen). TaqMan gene-specific assay primers and probes (Integrated DNA Technologies) were used together with TaqMan™ Fast Universal PCR Master Mix (2X) (Applied Biosystems). Information related to primer/probe sets is found below in Table 1. Samples were amplified as follows: denaturation at 95° C. for 20 sec followed by 50 cycles at 95° C. for 1 s and 60° C. for 20 s. The mRNA expression levels of cellular genes were normalized with that of TBP, as it showed to have the smallest standard deviation amongst a panel of housekeeping genes.

TABLE 1
Assays used to quantify the expression of various target genes.

| Gene | IDT assay | Gene | IDT assay |
|---|---|---|---|
| KRT5 | Hs.PT.58.14446018 | MUC5AC | |
| ITGA6 | Hs.PT.58.453862 | KRT18 | Hs.PT.58.4200217 |
| Foxj1 | Hs.PT.58.40371261 | Tslp | Hs.PT.58.22464960 |
| TUBB4B | Hs.PT.58.15334509.g | II25 | Hs.PT.58.39225615 |
| SCGB1A1 | Hs.PT.58.1190800 | FOXI1 | Hs.PT.58.40514934 |
| Neurogenin | Hs.PT.58.19734677.g | TMPRSS2 | Hs.PT.58.39408998 |
| 3 | | | |
| ASCL1 | Hs.PT.56a.23464.gs | TBP | Hs.PT.39a.22214825 |
| ACE2 | Hs.PT.58.27645939 | | |

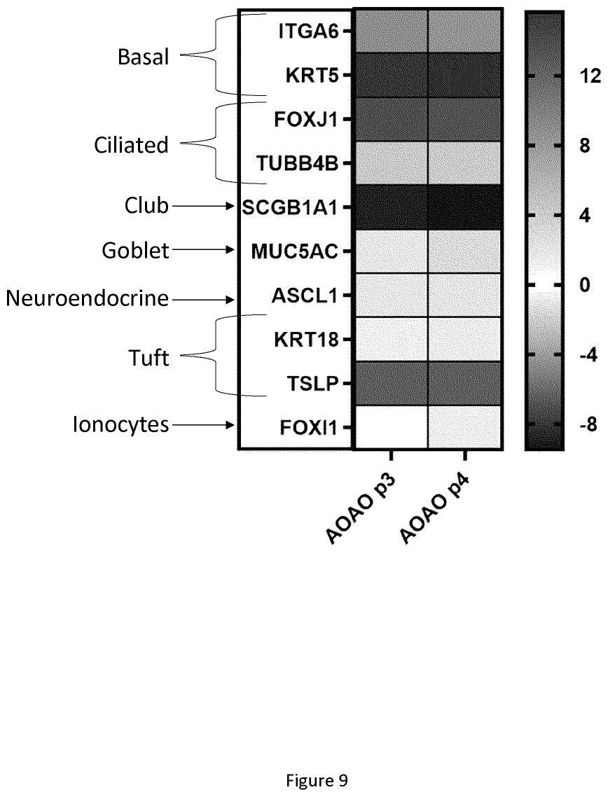

The expression of the markers outlined above was assessed in apical-out organoids formed in an apical-out organoid medium. As controls, the expression of these markers was also assessed in cells that were formed using the Airway Organoid Kit (STEMCELL Technologies), in cells grown at air-liquid interface using PneumaCult™ ALI (STEMCELL Technologies), and in cells expanded using PneumaCult™ Ex Plus (STEMCELL Technologies). The results of the analysis are shown in FIG. 9, and expression was normalized to Tata-binding protein (TBP).

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method of forming apical-out organoids in a culture, comprising:
contacting a population of cells with an organoid medium comprising an inhibitor of notch signaling and/or an inhibitor of signaling through a TGF; and
culturing the population of cells in the organoid medium and in the absence of an added extracellular matrix or extracellular matrix protein to obtain apical-out organoids,
wherein an apical surface of at least a portion of the apical-out organoids faces away from a core thereof.

2. The method of claim 1, wherein the population of cells are pulmonary lineage cells, preferably bronchial or nasal epithelial cells

3-4. (canceled)

5. The method of claim 1, further comprising aggregating the population of cells.

6. The method of claim 5, wherein aggregating the population of cells is in the organoid medium.

7. The method of claim 5, wherein aggregating the population of cells is for between 1 and 7 days and wherein aggregating comprises depositing between 10 and 2000 single cells or cells comprised in clumps into a microwell device.

8-9. (canceled)

10. The method of claim 1, wherein the organoid medium is serum-free.

11. The method of claim 1, wherein the organoid medium comprises the inhibitor of signaling through a TGF.

12. The method of claim 11, wherein the TGF is TGFbeta.

13-15. (canceled)

16. The method of claim 1, wherein culturing the population of cells is under non-adherent conditions.

17. (canceled)

18. The method of claim 1, wherein at least 25% of cells of the portion of the organoids are ciliated.

19. (canceled)

20. The method of claim 1, wherein among at least 60% of the organoids the apical surface thereof is in contact with the organoid medium.

21. (canceled)

22. The method of claim 1, wherein the organoids express one or more markers of ciliogenesis and the one or more markers of ciliogenesis comprise FOXJ1 and/or TUBB4B.

23. (canceled)

24. The method of claim 1, wherein the organoids express one or more of TJP1 (i.e. ZO-1) and ACE2.

25. The method of claim 1, wherein the organoids do not express MUC5AC.

26. The method of claim 1, wherein the organoids support the replication of viruses.

27. (canceled)

28. An organoid medium for generating apical-out organoids, the medium comprising a basal medium and an inhibitor of notch signaling and/or an inhibitor of signaling through a TGF.

29. The organoid medium of claim 28, wherein the organoid medium is serum-free.

30. The organoid medium of claim 28, wherein the organoid medium does not contain or come into contact with an added extracellular matrix or extracellular matrix protein.

31. The organoid medium of claim 28, further comprising the inhibitor of signaling through a TGF.

32. The organoid medium of claim 31, wherein the TGF is TGFbeta.

33-37. (canceled)
